SEQUENCE LISTING (10 sequences)

SEQ ID NO: 1 - DNA, 474 bp, synthetic construct
  misc_feature 1..474: note = Murine PD-L1-T epitope sequence
  source 1..474: mol_type = other DNA
ggatcctttatgacctattggcatctgctgaacgcgtttaccgtgaccgtgccgaaagacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaacgtgattctgggcgcgattctgctgtgcctgggcgtggcgctgacctttatttttcgcctgcgcaaaggctaagtcgac

SEQ ID NO: 2 - protein, 153 aa, synthetic construct
  REGION 1..153: note = Murine PD-L1-T epitope amino acid sequence
FMTYWHLLNAFTVTVPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINVILGAILLCLGVALTFIFRLRKG

SEQ ID NO: 3 - protein, 17 aa, synthetic construct
  REGION 1..17: note = PD-Long1
FMTYWHLLNAFTVTVPK

SEQ ID NO: 4 - protein, 23 aa, synthetic construct
  REGION 1..23: note = PD-Long2
VILGAILLCLGVALTFIFRLRKG

SEQ ID NO: 5 - protein, 113 aa, Canis lupus
DLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNIS

SEQ ID NO: 6 - protein, 113 aa, Felis catus
DLYVVEYGSNVTMECRFPVEEQLDLVSLIVYWEMEDKKIIQFVQGKEDLKVQHRSYSQRAQLLKDQLFLGKAALQITNVTLEDAGVYCCLIGYGGADYKRITLKVHAPYRKIN

SEQ ID NO: 7 - protein, 113 aa, synthetic construct
  REGION 1..113: note = PD-L1 Homo sapiens variant 1
RILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLG

SEQ ID NO: 8 - protein, 113 aa, synthetic construct
  REGION 1..113: note = PD-L1 Homo sapiens variant 2
DLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKIN

SEQ ID NO: 9 - protein, 113 aa, synthetic construct
  REGION 1..113: note = murine PD-L1 amino acid sequence
DLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKIN

SEQ ID NO: 10 - DNA, 360 bp, synthetic construct
  misc_feature 1..360: note = murine PD-L1 sequence
  source 1..360: mol_type = other DNA
ggatccggtaccgacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaactaagtcgac

(19) United States
(12) Patent Application Publication
(10) Pub. No.: US 2024/0325510 A1
(43) Pub. Date: Oct. 3, 2024

(54) Novel anti-cancer vaccine composition and a method of vaccination using the same

(71) Applicant: EULJI UNIVERSITY INDUSTRY ACADEMY COOPERATION FOUNDATION, Seongnam-si (KR)

(72) Inventors: Seung Hoon LEE, Daejeon (KR); Yun Hee KANG, Daejeon (KR); Myung Shin LEE, Daejeon (KR); Seung Ro HAN, Daejeon (KR)

(21) Appl. No.: 18/668,477

(22) Filed: May 20, 2024

Related U.S. Application Data
(63) Continuation of application No. PCT/KR2022/018361, filed on Nov. 18, 2022.

(30) Foreign Application Priority Data
Nov. 19, 2021 (KR) 10-2021-0160382

(51) Int. Cl. A61K 39/00; A61P 35/00; C12N 1/20; C12N 15/74; C12R 1/245; C12R 1/46

(52) U.S. Cl. CPC A61K 39/001111; A61P 35/00; C12N 1/205; C12N 15/746; A61K 2039/522; A61K 2039/523; C12R 2001/245; C12R 2001/46

(57) ABSTRACT

The present invention provides an anti-cancer vaccine composition and method of vaccination using the same, which can effectively inhibit the development and growth of various cancers including colorectal cancer, by inducing vaccination through the expression of PD-L1 or PD-L1-T epitope proteins on the surface of strains of the genus Lactobacillus.

### CROSS-REFERENCES TO RELATED APPLICATIONS

The present application claims priority to and benefit from Korean Patent Application KR10-2021-0160382, filed Nov. 19, 2021, which is incorporated herein by reference in its entirety, and also claims priority to and benefit from International Patent Application PCT/KR2022/018361, filed Nov. 18, 2022.

### ACKNOWLEGEMENT OF THE GOVERNMENT-FUNDED RESEARCH PROJECTS

The present invention was made with government support of Creative Challenges Research-Based Support Project under Grant No. RS-2023-00246807 entitled “Development of a dual-targeted cancer vaccine using bacteria expressing immune checkpoint proteins” (Research Period: Jun. 1, 2023˜May 31, 2026, Lead Research Institute: Eulji University Industry Academy Cooperation Foundation) and Local University Outstanding Scientist Support Project under Grant No. NRF-2020R1I1A3066989 (Research Period: Jun. 1, 2020˜May 31, 2023, Lead Research Institute: Eulji University Industry Academy Cooperation Foundation) awarded by Ministry of Education of Republic of Korea.

### TECHNICAL FIELD

The present invention relates to a novel anti-cancer vaccine composition and, more particularly, to a novel anti-cancer vaccine composition comprising PD-L1 or PD-L1-T epitope proteins expressed on the surface of strains of the genus Lactobacillus and methods of vaccination using the same.

### BACKGROUND ART

PD-L1 is a protein on the surface of cancer cells or hematopoietic cells that binds to Programmed Death-1 (PD-1) receptors and activates a negative regulatory signaling pathway that inhibits the proliferation and expression of T-cells (Freeman et al., J. Exp. Med., 192:1027-1034, 2000). Hence, it is also called PD-1 ligand 1 (PD-L1 or CD274). Human PD-L1 gene encodes a full-length protein of 290 amino acid residues (NCBI accession NP_054862.1), and the PD-L1 protein contains a major peptide that is expressed on the cell surface and then cleared. PD-L1 is expressed on human heart, lung, thymus, and vascular endothelial cells, and is expressed at low levels in many other tissues and cell types in the body including Antigen Presenting Cells, Peripheral Blood Monocytes, and other immune cells (Freeman et al., J. Exp. Med., 192: 1027-1934; 2000, Eppihimer et al., Microcirculation, 9:133-145, 2002). Most cell types express high levels of PD-L1 when stimulated by IFN-γ, IL-12, and type I interferons. More recently, PD-L1 has been reported to have a specific interaction with B7-1 (a member of the B7 family called CD80) instead of binding to PD-1 (Butte et al., Immunity 27:111-122, 2007). The interaction of PD-L1 and CD80 negatively regulated the function and activity of T cells, while blocking of the interaction of PD-L1 and CD80 in mice enhanced the immune response to OVA antigens. Therefore, simultaneously blocking the binding of PD-L1 to PD-1 and CD80 may have a synergistic effect on the treatment of cancer and viral infections. In this regard, Korean Patent Publication Gazette No. 2018-0037222 discloses a novel anti-PD-L1 antibody.

### DETAILED DESCRIPTION OF THE INVENTION

### Technical Problem

However, the above prior art relates to the treatment of tumors using anti-PD-L1 antibodies, and inhibition of tumor development and growth by inducing PD-L1 vaccination in a host such as a mouse is still an unexplored field.

The present invention is intended to address the above and other problems, and aims to provide a novel anti-cancer vaccine composition and method of vaccination using the same, which can effectively inhibit the development and growth of various cancers including colorectal cancer, by inducing vaccination through the expression of PD-L1 or PD-L1-T epitope proteins on the surface of strains of the genus Lactobacillus. However, these tasks are exemplary and the scope of the present invention is not limited thereto.

### Technical Solution

In one aspect of the present invention, there is provided a recombinant bacterium Etransformed to present on its surface a PD-L1 protein, or a fusion protein in which the PD-L1 protein is linked to a T epitope.

In another aspect of the present invention, there is provided an anti-cancer vaccine composition comprising the recombinant bacterium as an active ingredient.

In another aspect of the present invention, there is provided a method of preventing and treating cancer, comprising administering the anti-cancer vaccine composition to an individual suffering from cancer.

### Effect of the Invention

The novel anti-cancer vaccine composition of the present invention, which acts as a vaccine by expressing PD-L1 protein or PD-L1-T epitope protein on the surface of strains of the genus Lactobacillus as described above, effectively inhibits the development and growth of tumors. Therefore, it can be used as a therapeutic agent for preventing recurrence of cancer, and can be expected to reduce treatment costs compared to conventional antibody treatment for immunotherapy of cancer. However, the scope of the present invention is not limited by these effects.

### BRIEF DESCRIPTION OF DRAWINGS

FIG. 1 is an illustration of the nucleotide sequence and amino acid sequence of PD-L1 from a mouse according to one embodiment of the present invention, showing the recognition site of a restriction enzyme added to the partial sequence of mouse PD-L1 used in the present invention to make the gene construct.

FIG. 2 is an illustration of the PD-L1-epitope fusion nucleotide sequence and amino acid sequence from a mouse according to one embodiment of the present invention, showing PD-Long1 (17 amino acids including a portion of HLA-A2), and PD-Long2 (23 amino acids including a portion of HLA class II) fused to the partial sequence of mouse PD-L1 and the restriction enzymes used to make the gene construct.

FIG. 3a is a schematic diagram illustrating the structure of a plasmid vector constructed to express mouse PD-L1 or PD-L1-T-epitope on the surface of Latobacillus casei (L. casei), according to one embodiment of the present invention.

FIG. 3b is a schematic diagram illustrating a gene construct prepared to express mouse PD-L1 or PD-L1-T-epitope on the surface of Latobacillus casei, according to one embodiment of the present invention.

FIG. 4 is a photograph showing the results of Western blot analysis of mPD-L1 antigen produced by PgsA-mPD-L1/L. casei and PgsA-mPD-L1-T-epitope/L. casei, according to one embodiment of the present invention. The combined antigens of PgsA anchor (42 kDa) and mPD-L1 (12 kDa) were identified at the 54 kDa site.

FIG. 5 is a schematic diagram illustrating the progression of experiments using CT26 cells as tumor cells for the preparation of BALB/c mice and xenograft tumor models in the present invention.

FIG. 6 is a schematic diagram illustrating the progression of experiments using MC38 cells as tumor cells for the preparation of C57BL/6 mice and xenograft tumor models in the present invention.

FIG. 7a is a graph representing the results of individual weight measurements of BALB/c mice administered with PgsA-mPD-L1/L. casei and PgsA-mPD-L1-T epitope/L. casei, respectively, according to one embodiment of the present invention.

FIG. 7b is a graph representing the average weight measurement results of BALB/c mice administered with PgsA-mPD-L1/L. casei and PgsA-mPD-L1-T epitope/L. casei in the present invention, respectively.

FIG. 8 is a series of histograms representing the flow cytometry results from a FACS analysis for the observation of PD-L1 expression in CT26 mouse colon cancer cell lines treated and stimulated with interferon-gamma (IFN-γ). PD-L1 expression was increased 7.1-fold by the interferon-gamma (IFN-γ) stimulation.

FIG. 9a is a graph representing the results of an ELISA analysis to verify the production of mPD-L1 antibodies in BALB/c mice after three times of immunizations with PgsA-mPD-L1/L. casei and PgsA-mPD-L1-T epitope/L. casei, respectively, according to one embodiment of the present invention. Successful mPD-L1 vaccination in BALB/c mice was observed.

FIG. 9b is a graph representing the results of an ELISA analysis to verify the production of mPD-L1 antibodies in BALB/c mice after three times of immunizations with PgsA-mPD-L1/L. casei and PgsA-mPD-L1-T epitope/L. casei, respectively, according to one embodiment of the present invention.

FIG. 10a is a graph representing the results of individual tumor growth analysis, verifying the inhibition of colon cancer development and growth in a BALB/c mouse model vaccinated with mPD-L1 or mPD-L1-T epitope, according to one embodiment of the present invention.

FIG. 10b is a graph representing the results of tumor growth curve analysis, verifying the inhibition of colon cancer development and growth in a BALB/c mouse model vaccinated with mPD-L1 or mPD-L1-T epitope, according to one embodiment of the present invention.

FIG. 10c is a photograph comparing the size of harvested tumors, verifying the inhibition of colon cancer development and growth in a BALB/c mouse model vaccinated with mPD-L1 or mPD-L1-T epitope, according to one embodiment of the present invention.

FIG. 10d is a graph representing the results of mean tumor volume analysis, verifying the inhibition of colon cancer development and growth in a BALB/c mouse model vaccinated with mPD-L1 or mPD-L1-T epitope, according to one embodiment of the present invention.

FIG. 10e is a graph representing the results of mean tumor weight analysis, verifying the inhibition of colon cancer development and growth in a BALB/c mouse model vaccinated with mPD-L1 or mPD-L1-T epitope, according to one embodiment of the present invention.

FIG. 11 is a microscopic image showing the observation of mPD-L1 expression in immunohistochemically stained tumor tissue harvested from a BALB/c mouse model vaccinated with mPD-L1 or mPD-L1-T epitope, according to an embodiment of the present invention.

FIG. 12a is a graph representing the results of individual body weight measurements of C57BL/6 mice administered with PgsA-mPD-L1/L. casei and PgsA-mPD-L1-T epitope/L. casei, respectively, according to one embodiment of the present invention.

FIG. 12b is a graph representing the results of average body weight measurements of C57BL/6 mice treated with PgsA-mPD-L1/L. casei and PgsA-mPD-L1-T epitope/L. casei, respectively, according to one embodiment of the present invention.

FIG. 13 is a series of histograms representing the results of FACS analysis for the observation of PD-L1 expression in MC38 mouse colon cancer cell lines treated and stimulated with interferon-gamma (IFN-γ). PD-L1 expression was increased 10.7-fold by the interferon-gamma (IFN-γ) stimulation.

FIG. 14a is a graph representing the results of an ELISA analysis to verify the production of mPD-L1 antibodies in C57BL/6 mice after three times of immunizations with PgsA-mPD-L1/L. casei and PgsA-mPD-L1-T epitope/L. casei, respectively, according to one embodiment of the present invention. Successful mPD-L1 vaccination of C57BL/6 mice was observed.

FIG. 14b is a graph representing the results of an ELISA analysis to verify the production of mPD-L1 antibodies in C57BL/6 mice after three times of immunizations with PgsA-mPD-L1/L. casei and PgsA-mPD-L1-T epitope/L. casei, respectively, according to one embodiment of the present invention.

FIG. 15a is a graph representing the results of individual tumor growth analysis, verifying the inhibition of colon cancer development and growth in a C57BL/6 mouse model vaccinated with mPD-L1 or mPD-L1-T epitope according to one embodiment of the present invention.

FIG. 15b is a graph representing the results of tumor growth curve analysis, verifying the inhibition of colorectal cancer development and growth in a C57BL/6 mouse model vaccinated with mPD-L1 or mPD-L1-T epitope according to one embodiment of the present invention.

FIG. 15c is a photograph comparing the size of harvested tumors, verifying the inhibition of colon cancer development and growth in a C57BL/6 mouse model vaccinated with mPD-L1 or mPD-L1-T epitope, according to one embodiment of the present invention.

FIG. 15d is a graph representing the results of mean tumor volume analysis to verify the inhibition of colon cancer development and growth in a C57BL/6 mouse model vaccinated with mPD-L1 or mPD-L1-T epitope according to one embodiment of the present invention.

FIG. 15e is a graph representing the results of mean tumor weight analysis to verify the inhibition of colon cancer development and growth in a C57BL/6 mouse model vaccinated with mPD-L1 or mPD-L1-T epitope according to one embodiment of the present invention.

FIG. 16 is a microscopic image showing the observation of mPD-L1 expression in immunohistochemically stained tumor tissue harvested from a C57BL/6 mouse model vaccinated with mPD-L1 or mPD-L1-T epitope, according to one embodiment of the present invention.

### BEST MODE FOR THE INVENTION

### Definitions of Terms

As used herein, the term “Lactobacillus casei” refers to an anaerobic microorganism of the genus Lactobacillus sp., which is highly resistant to low pH conditions, acid and bile, and exhibits excellent adhesion to human epithelial cell lines.

As used herein, the term “Programmed Death-Ligand 1 (PD-L1)” refers to a protein on the surface of cancer cells or hematopoietic cells that was originally cloned from a B7 protein family member (B7-H1) (Dong et al., Nature Med., 5: 1365-1369, 1999). It is therefore also referred to as CD274 or B7-H1. The calculated molecular weight of the full-length PD-L1 is 33 kDa, but the molecular weight observed on Western blots by glycosylation is 50 kDa. When PD-L1 and PD-L2 which are proteins on the surface of cancer cells bind to PD-1, which is a protein on the surface of T cells, the T cells are unable to attack the cancer cells. Immune checkpoint inhibitors, such as anti-PD-1 antibodies or anti-PD-L1 antibodies, bind to PD-1 on the T cells or PD-L1 on the cancer cells and inhibit the immune evasion of cancer cells. The PD-L1 (B7-H1) and PD-L2 (B7-DC) sequences identified so far are known to react with PD-1 to induce negative signal transduction and inhibition of TCR- and CD28-mediated T cell activity, growth factors, and cytokines such as IL-2 or IFN-7 (Riley et al., Immunol. Rev., 229(1): 114-125, 2009).

As used herein, the term “T epitope” refers to a PD-Long1 (SEQ ID NO: 3) peptide comprising 17 amino acids including an HLA-A2 sequence derived from a PD-L1 precursor, and a PD-Long2 (SEQ ID NO: 4) peptide comprising 23 amino acids including an HLA class II sequence, for inducing a cytotoxic T lymphocyte (CTL) immune response (S. Munir Ahmad et al., Oncoimmunol., 5(8):e1202391, 2016).

### A Detailed Description of the Invention

In one aspect of the present invention, there is provided a recombinant bacterium transformed to present on its surface a PD-L1 protein or a fusion protein in which the PD-L1 protein is linked to a T epitope.

In the recombinant bacterium, the PD-L1 protein or the fusion protein may be presented on the surface by binding to a PgsA anchor and may be inserted into multiple cloning sites of the pKV-Pald-PgsA380L vector. Furthermore, the PD-L1 may be a mammalian-derived PD-L1, where the mammal may be a primate, carnivore, proboscidean, artiodactyl, perissodactyl, or rodent, or, may be selected from the group consisting of primates including humans; carnivores including dogs (SEQ ID NO: 5), lions, tigers, and cats (SEQ ID NO: 6); rodents including brown rats, hamsters, mice (SEQ ID NO: 9) and guinea pigs; lagomorphs including hares and pikas; perissodactyls including horses, donkeys, rhinoceroses, and tapirs; artiodactyls including cattle, deer, long-tailed gorals, sheep, and antelopes; and proboscideans including elephants; but preferably human PD-L1 (SEQ ID NO: 7 or 8). The SEQ ID NO: 7 represents a human PD-L1 variant sequence 1, SEQ ID NO: 8 represents a human PD-L1 variant sequence 2, and SEQ ID NO: 10 represent a mouse PD-L1 nucleic acid sequence.

In the recombinant bacterium, the bacterium may be non-pathogenic bacterium genus selected from the group consisting of Lactobacillus sp., Parabacteroides sp., Streptococcus sp., Bifidobacterium sp., Lactococcus sp., and Enterococcus sp.

In the recombinant bacterium, the PD-L1 protein may comprise an amino acid sequence selected from the group consisting of SEQ ID NOs: 2 to 6 and 9.

In another aspect of the present invention, there is provided an anti-cancer vaccine composition comprising the recombinant bacterium as an active ingredient.

The anti-cancer vaccine composition may have anti-cancer activity against Breast cancer, Lung cancer, Colorectal cancer, Gastric cancer, Bladder cancer, Pancreatic cancer, Prostate cancer, or diffuse large B-cell lymphoma.

In another aspect of the present invention, there is provided a method of preventing and treating cancer, comprising administering the anti-cancer vaccine composition to an individual suffering from cancer. The individual may be any animal, including a human, having or having developed a cancerous disease, preferably a mammal, and more preferably a mammal other than a human.

The bacterium may be a member of the genus Lactobacillus sp., Parabacteroides sp., Streptococcus sp., Bifidobacterium sp., Lactococcus sp., or Enterococcus sp. In this case, the Lactobacillus sp. The bacterium of the genus Lactobacillus sp. may be Lactobacillus acidophilus, Lactobacillus rhamnosus, Lactobacillus bulgaricus, Lactobacillus casei, Lactobacillus gasseri, Lactobacillus helveticus, Lactobacillus fermentum, Lactobacillus paracasei, Lactobacillus reuteri, Lactobacillus salivarius, or Lactobacillus plantarum. Further, the bacterium of the genus Enterococcus sp. may be Enterococcus faecium or Enterococcus faecalis, and the bacteria of the genus Lactococcus sp. may be Lactococcus lactis. Furthermore, the bacterium of the genus Streptococcus sp. may be Streptococcus thermophilus, and the bacterium of the genus Bifidobacterium sp. may be Bifidobacterium bifidum, Bifidobacterium breve, Bifidobacterium longum, or Bifidobacterium lactis. However, the term “bacterium” as used herein is not limited thereto.

The anti-cancer vaccine composition of the present invention may have activity against lung cancer, stomach cancer, liver cancer, bone cancer, pancreatic cancer, gallbladder cancer, cholangiocarcinoma, skin cancer, head and neck cancer, skin melanoma, uterine cancer, ovarian cancer, rectal cancer, colon cancer, colorectal cancer, breast cancer, uterine sarcoma, fallopian tube carcinoma, endometrial carcinoma, cervical carcinoma, vaginal carcinoma, vulvar carcinoma, esophageal cancer, laryngeal cancer, small intestine cancer, or thyroid cancer, and preferably a tumor or cancer associated with the interaction of PD-1/PD-L1, and more preferably a breast cancer, lung cancer, colorectal cancer, gastric cancer, bladder cancer, pancreatic cancer, prostate cancer, or diffuse large B-cell lymphoma.

The pharmaceutical composition of the present invention may comprise one or more pharmaceutical diluents selected from, but not limited to, brine, buffered saline, dextrose, water, glycerol, and ethanol. The pharmaceutical composition may be adapted for different purposes of administration and different diseases. The actual amount of active ingredient to be administered should be determined by considering various relevant factors, such as the disease to be treated, the severity of the patient's condition, co-administration with other agents (e.g., injectable agents), the patient's age, sex, weight, food, time of administration, route of administration, and the administration ratio of the composition. The composition may be administered once daily or in 1-3 divided doses, although the dosage and route of administration may be adjusted depending on the form and severity of the disease.

The pharmaceutical composition of the present invention may comprise a pharmaceutically acceptable carrier, and may further comprise pharmaceutically acceptable adjuvants, excipients or diluents in addition to the carrier. As used herein, the term “pharmaceutically acceptable” refers to a composition that is physiologically acceptable and does not normally cause gastrointestinal disturbances, allergic reactions such as dizziness, or similar reactions when administered to humans. Examples of such carriers, excipients, and diluents include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. The pharmaceutical composition of the present invention may further comprise fillers, anti-flocculants, lubricants, wetting agents, flavors, emulsifiers, and preservatives. Moreover, the pharmaceutical composition of the present invention may be formulated using methods known in the art to allow for rapid, sustained, or delayed release of the active ingredient upon administration to a mammal. Formulations include powders, granules, tablets, emulsions, syrups, aerosols, soft or hard gelatin capsules, sterile injectable solutions, and sterile powder forms.

The pharmaceutical composition of the present invention can be administered orally or parenterally. Parenteral administration refers to the administration of an agent by a route of administration other than oral, such as rectal, intravenous, peritoneal and intramuscular, arterial, transdermal, nasal, inhalation, ocular, and subcutaneous. Furthermore, the pharmaceutical composition may be formulated in any form, such as oral dosage forms, injectable solutions, or topical preparations. Moreover, the pharmaceutical composition of the present invention can be administered at a dose of 0.1 mg/kg to 1 g/kg, more preferably at a dose of 1 mg/kg to 600 mg/kg. Meanwhile, the dosage may be appropriately adjusted according to the age, gender and condition of the patient.

PD-L1 overexpression in cancer cells has been reported in a variety of cancers involving different types of tissues, and organs such as lung (Konishi et al., Clin. Cancer Res., 10: 5094-5100, 2004), liver (Shi et al., Int. J. Cancer, 128: 887-896, 2008), stomach (Wu et. al., Acta Histochem., 108: 19-24, 2006), kidney (Thompson et al., Proc. Natl. Acad. Sci. USA, 101: 17174-17179, 2004), breast (Ghebeh et al., Neoplasia, 8:190, 2006), ovary (Hamanishi et al., Proc. Natl. Acad. Sci. USA, 104: 3360-3365, 2007), pancreas (Nomi et al., Clin. Cancer Res. 13: 2151-2157, 2007), melanocytes (Hino et al., Cancer, 116: 1757-1766, 2010), and esophagus (Ohigashi et al., Clin. Cancer Res. 11: 2947-2953, 2005). The increased expression of PD-L1 in cancer is associated with poor prognosis of patient survival.

Blocking the binding of PD-L1 to PD-1 by anti-B7-H1 antibodies or anti-PD-L1 antibodies stimulates the proliferation and functional activity of T-cells and enhances the immune response to tumor growth and viral infection (Iwai et al., Proc. Natl. Acad. Soc. USA, 99: 12293-12297, 2002). Consistent with these studies, inhibition of PD-L1/PD-1 signaling activates the immune response against cancer cell growth, viral infection and spread in human bodies. HBV and HCV, which are common hepatitis viruses, induce overexpression of PD-1L in hepatocytes and activate PD-1 signaling in T effector cells, leading to T cell exhaustion and immune tolerance of viral infection (Boni et al., J. Virol., 8: 4215-4225, 2007). Similarly, HIV infection evades the human immune system by a similar mechanism. It has been reported that the therapeutic modulation of PD-L1/PD-1 signaling induced by antagonist molecules can restore and reactivate immune cell tolerance to block cancer and chronic viral infections (Blank et al., Cancer Immunol. Immunother., 54(4): 307-314, 2005). To date, most studies have been conducted on tumor treatment methods using anti-PD-L1 antibodies, but only a limited number of studies have explored inducing PD-L1 vaccination in hosts such as mice and its effects on tumor development and growth inhibition. Therefore, the present inventors prepared an antigen where PD-L1 (Programmed Death-Ligand 1) or PD-L1-T epitope was expressed on the surface of Lactobacillus casei, an E. coli bacterium. The present invention was completed by injecting the antigen into mice for vaccination and observing that the development and growth of colon cancer were effectively inhibited by the injection of colon cancer cell lines, CT26 and MC38, into the syngeneic mouse models (BALB/c, C57BL/6). The inhibitory effect of tumor development and growth through the PD-L1 or PD-L1-T epitope vaccination method of the present invention may provide a combination treatment method for preventing the occurrence of solid cancers, including colon cancer, and their recurrence after surgery.

Unlike previous patents that provide therapeutic methods using PD-1 and PD-L1 antibodies for immunotherapy of cancer targeting immune checkpoint proteins, the present invention is the first to be reported in Korea and abroad as a therapeutic method for inhibiting tumor development and growth and preventing recurrence of the cancer after surgery through vaccination of PD-L1 or PD-L1-T epitope in a host (mammals including humans and dogs).

The present invention will now be described in more detail with reference to the following examples. However, the invention is not limited to the embodiments disclosed herein, but may be embodied in many different forms, and the following examples are provided to make the disclosure of the invention complete and to give those of ordinary skill in the art a complete idea of the scope of the invention.

### Example 1: Preparation of mPD-L1 Antigens and Vectors

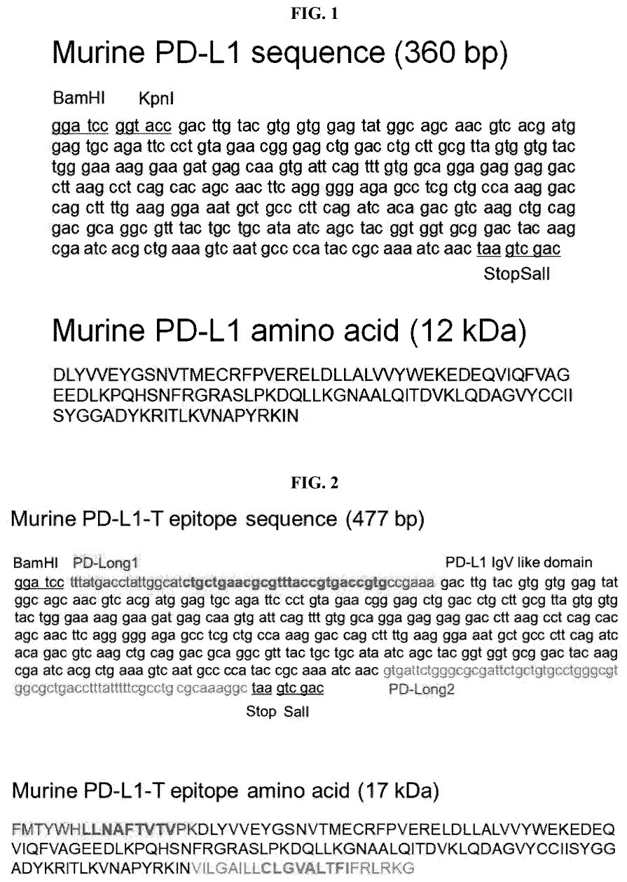

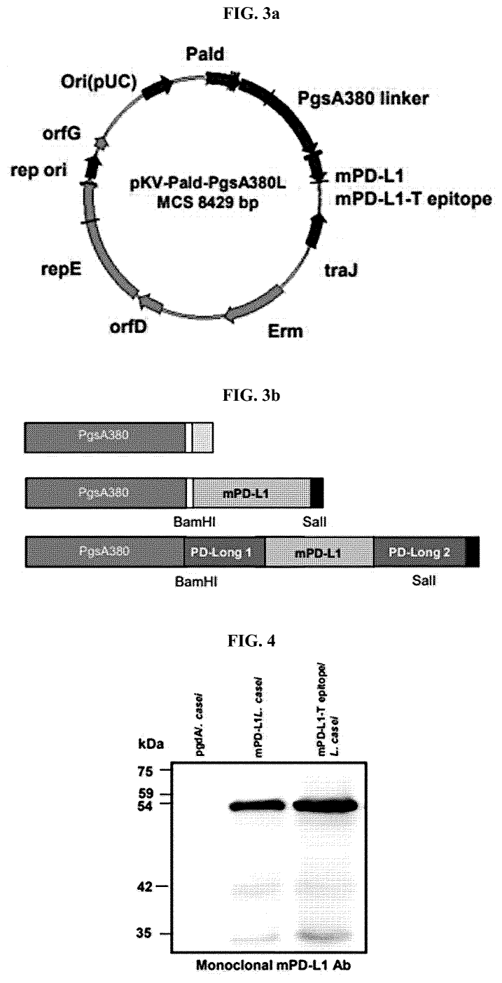

The present inventors prepared a vector and mPD-L1 antigen for expressing mouse PD-L1 protein on the surface of a Lactobacillus casei strain. Specifically, the mPD-L1 antigen for expressing mouse PD-L1 on the surface of Lactobacillus casei strain was provided by the laboratory of Prof. Chul-Jung Kim, College of Veterinary, Chungnam National University. The mouse PD-L1 gene consists of a 360 bp sequence and encodes a protein with a molecular weight of 12 kDa (FIG. 1). The mouse PD-L1 sequence was synthesized and inserted into the multicloning site of the pKV-Pald-PgsA380L vector (Bioleaders, Korea) to prepare a vector for expressing mouse PD-L1 on the surface of the Lactobacillus caseil strain (FIG. 3). After introducing the vector into the strain, the transformed strain was cultured to express the mouse PD-L1 on the surface of the strain, which was later inactivated to prepare an antigen that can be injected into mice. The mPD-L1 antigen produced using the above-prepared PgsA-mPD-L1/L. casei was analyzed by western blot, and an antigen comprising a PgsA anchor (42 kDa) and mPD-L1 (12 kDa) bound to each other was identified at the 54 kDa site (FIG. 4).

### Example 2: Preparation of mPD-L1-T-Epitope Antigens and Vectors

The present inventors prepared a vector for expressing mouse PD-L1-T epitope protein on the surface of a Lactobacillus casei strain and a gene construct encoding an mPD-L1-T epitope fusion protein. Specifically, the PD-L1-T epitope antigen made by expressing the PD-L1-T epitope on the surface of Lactobacillus casei strain was provided by the laboratory of Prof. Chul-Jung Kim, College of Veterinary, Chungnam National University. The mouse PD-L1-T epitope consists of a sequence of 477 bp and is a protein with a molecular weight of 17 kDa. It was prepared as a T epitope to maximize the T cell response of the PD-L1 antigen, wherein PD-Long1 (17 amino acids including 9-mer of HLA-A2 sequence, SEQ ID NO: 3) and PD-Long2 (23 amino acids including 15-mer of HLA class II sequence, SEQ NO: 4) peptides were expressed together with mouse PD-L1 (FIG. 2).

The above mouse PD-L1-T epitope sequence was synthesized and inserted into the multicloning site of the pKV-Pald-PgsA380L vector to prepare a vector for expressing the mouse PD-L1-T epitope on the surface of a Lactobacillus casei strain (FIG. 3). After the vector was introduced into the above strain, the transformed L. casei was cultured to express the mouse PD-L1-T epitope on the surface of L. casei, which was later inactivated to prepare an antigen that can be injected into mice. The mouse PD-L1-T epitope antigen (59 kDa) produced using the above-prepared PgsA-mPD-L1-T epitope/L. casei was analyzed by western blot, and the antigen of PgsA anchor (42 kDa) and mPD-L1-T epitope (17 kDa) bound to each other was identified at a position almost similar to that of the mouse PgsA anchor-PD-L1 antigen (54 kDa) (FIG. 4).

### Example 3: Mouse Animal Model Manufacturing

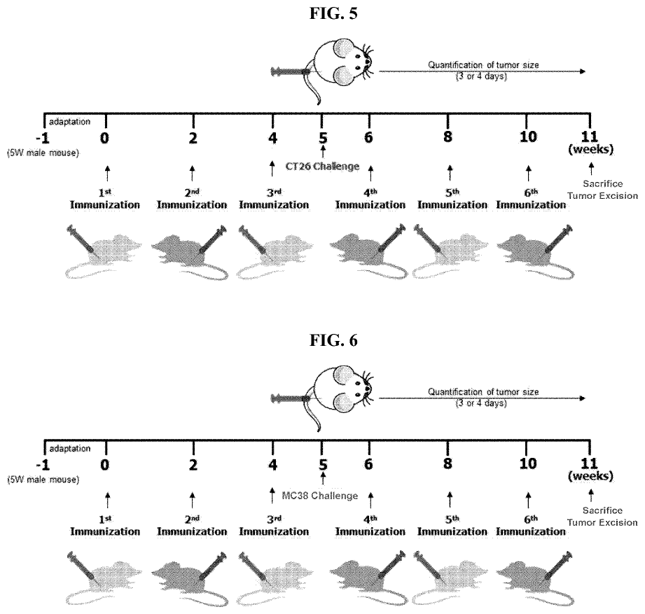

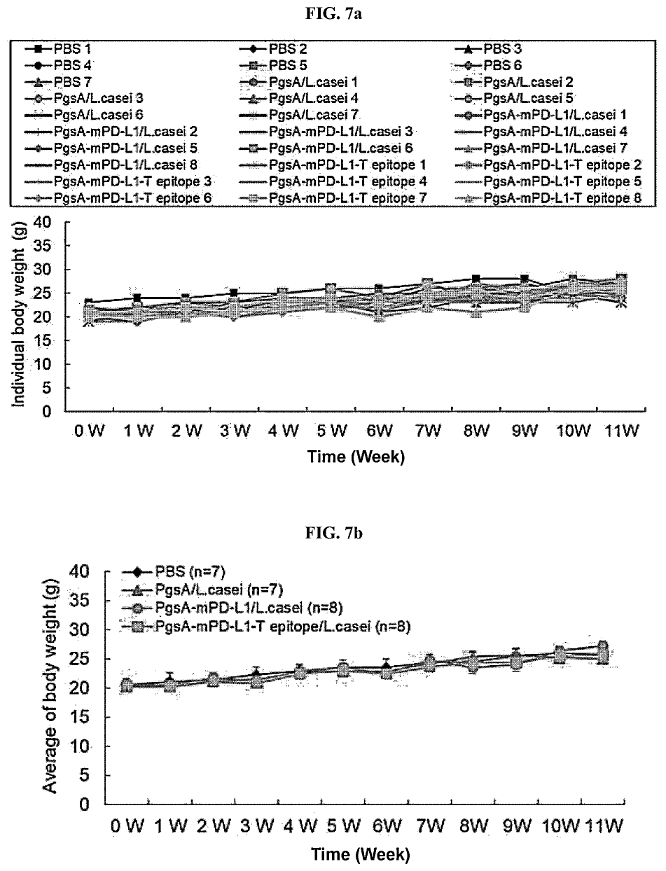

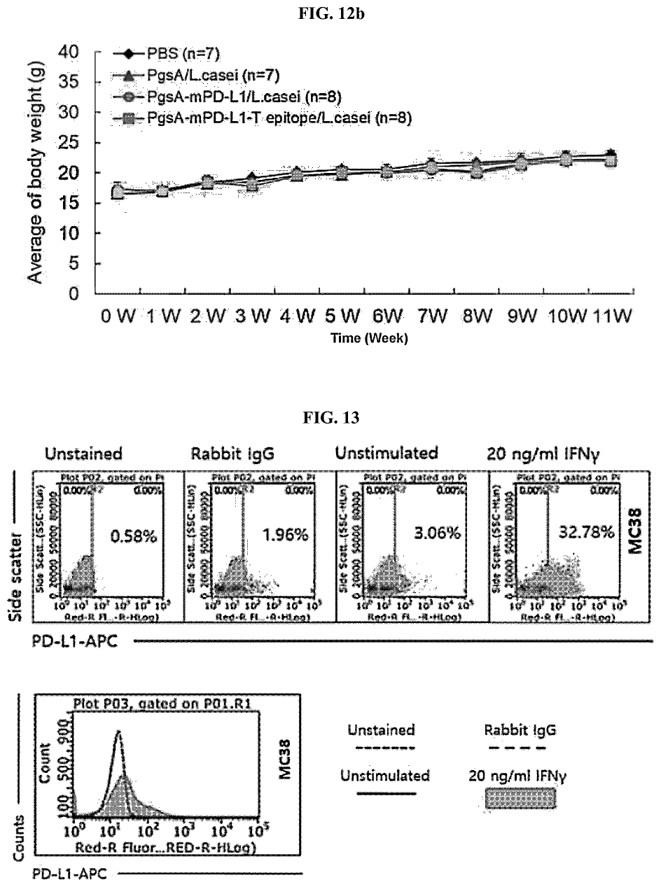

The present inventors prepared two types of mouse animal models for the mPD-L1 vaccination. Specifically, the mPD-L1/L. casei antigen and mPD-L1-T-epitope/L. casei antigen prepared in Examples 1 and 2, respectively, were applied to two types of mice (BALB/c and C57BL/6) as follows. First, 5-week-old male BALB/c mice and 5-week-old female C57BL/6 were purchased from KOATEC (Gyeonggi-do, Korea), and animal experiments were conducted in accordance with the animal experimentation guidelines of the Ministry of Food and Drug Safety after approved by the Eulji University Animal Care and Use Committee (approval number EUIACUC18-29). The male BALB/c and female C57BL/6 mice were categorized into PBS group (control), PgsA/L. casei group, PgsA-mPD-L1/L. casei group, PgsA-mPD-L1-T-epitope/L. casei group, and PgsA-mPD-L1-T-epitope/L. casei group, with four mice put together per case, and were acclimatized to the new environment for one week with water and chow. Then, at the age of 6 weeks, the antigens prepared for each group were intramuscularly injected into the thighs of the hind legs of the mice, and the injection was conducted per every two weeks to produce mPD-L1 vaccinated mouse models. In particular, the left and right hind legs of the mice were alternately injected during the intramuscular injections every two weeks (FIGS. 5 and 6). The weight changes of the mice in each group were measured every week during the period of producing the above mouse animal models. As a result, both of the BALB/c and C57BL/6 mice showed little difference in body weight between the control and experimental groups during the experimental period (FIGS. 7a, 7b, 12a and 12b). The above results suggest that mPD-L1 vaccination with the mPD-L1/L. casei antigen or mPD-L1-T-epitope antigen of the present invention is not harmful to mice.

### Example 4: Preparation of Colon Cancer Cells

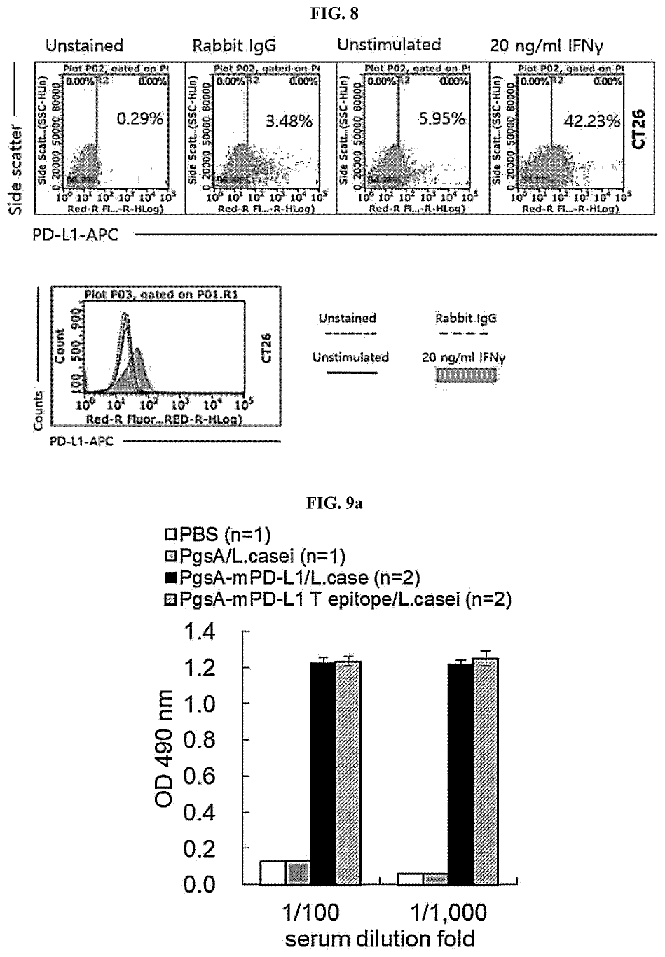

The present inventors prepared mouse colon cancer cells, CT26 and MC38, for transplanting into the two types of vaccinated mouse models, mPD-L1 or mPD-L1-T epitope mice (BALB/c and C57BL/6), prepared according to one embodiment of the present invention. Specifically, the mouse colon cancer cell lines CT26 and MC38 were seeded in 5×10⁵ cells/T75 culture flasks, treated with 20 ng/ml of mouse interferon-gamma after 1 day, and stimulated for 3 days to increase the expression of PD-L1. The colon cancer cells with increased PD-L1 expression by interferon-gamma were stained with anti-PD-L1 antibodies for 30 minutes, followed by conjugation with APC (fluophore)-coupled IgG secondary antibodies for 30 minutes, and then subjected to FACS analysis. As a result, the CT26 cell line showed a 7.1-fold increase in PD-L1 expression in the interferon-gamma-treated group compared to the untreated group (FIG. 8), and similarly, the MC38 cell line showed a 10.7-fold increase in PD-L1 expression by the interferon-gamma treatment (FIG. 13). Furthermore, histogram analysis confirmed the increase in PD-L1 expressing cells among the cells treated with the interferon-gamma stimulation. The present inventors then prepared CT26 and MC38 cells with increased PD-L1 expression by interferon-gamma stimulation at 1×10⁶ cells/mL, and injected them at a dose of 100 μl per mouse for animal testing.

### Example 5: Verification of Vaccination

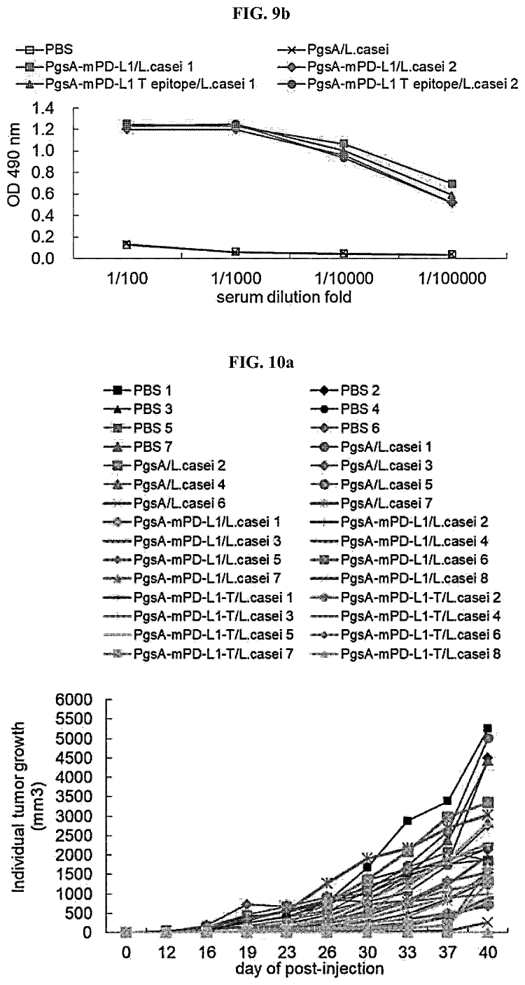

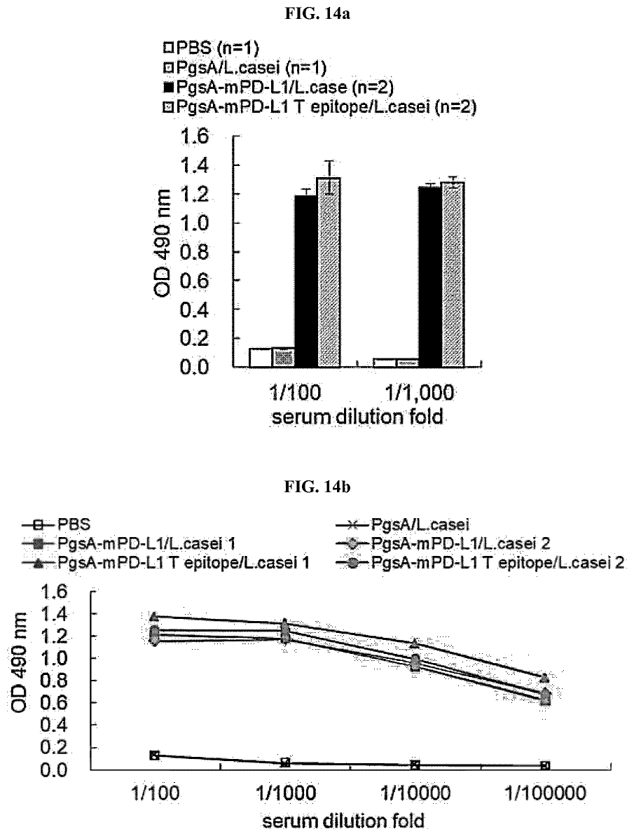

To determine whether vaccination by the mPD-L1 or mPD-L1-T-epitope according to one embodiment of the present invention went well in the mouse models, the present inventors collected blood from each group of mice 4 days after the third antigen immunization and allowed it to stand at room temperature for 30 minutes, then centrifuged it at 13,000 rpm for 30 minutes to separate the serum-containing supernatant from the blood. The separated serum was then sequentially diluted from 1:100 to 1:100000 and applied to ELISA plates coated with mPD-L1 antigens, allowed to bind at room temperature for 2 hours. The plate was then washed three times with TBST buffer and treated with HRP-conjugated IgG secondary antibodies, allowed to react at room temperature for 1 hour. Subsequently, the plate was washed three times again with the TBST buffer and treated with OPD chromogenic reagent to develop color in the sample where the antigen-antibody reaction took place. Here, the reaction was stopped by treating with sulfuric acid. Afterward, the results were analyzed for optical density using an OD₄₉₀ nm ELISA Reader, and the degree of mPD-L1 or mPD-L1-T-epitope vaccination in each group was compared. The results showed that the mice in the PBS and PgsA/L. casei groups were not vaccinated, but the mice in the PgsA-mPD-L1/L. casei group and the PgsA-mPD-L1-T-epitope group of the present invention were well immunized with mPD-L1 antibodies (FIGS. 9a, 9b, 14a and 14b).

### Example 6: Validation of Inhibition of Colon Cancer Development and Growth

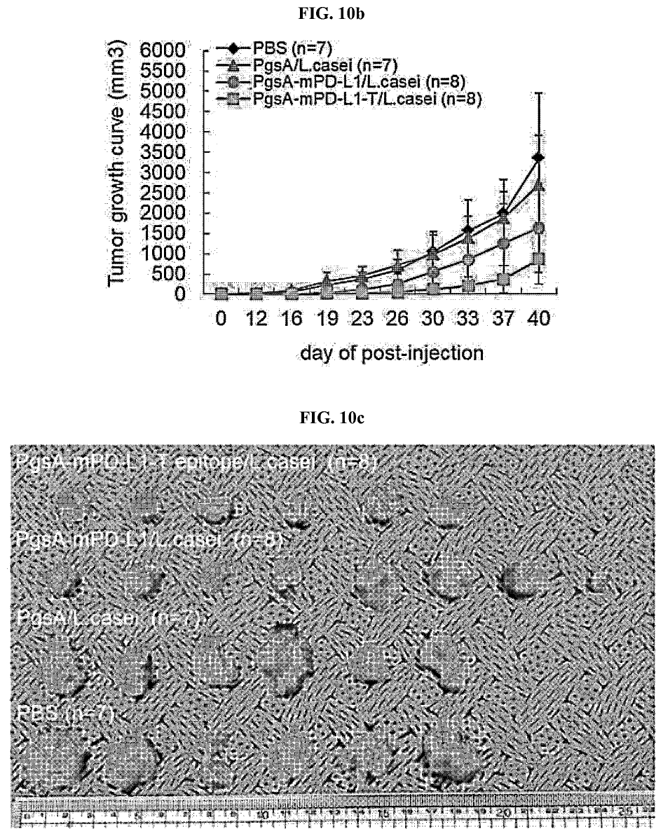

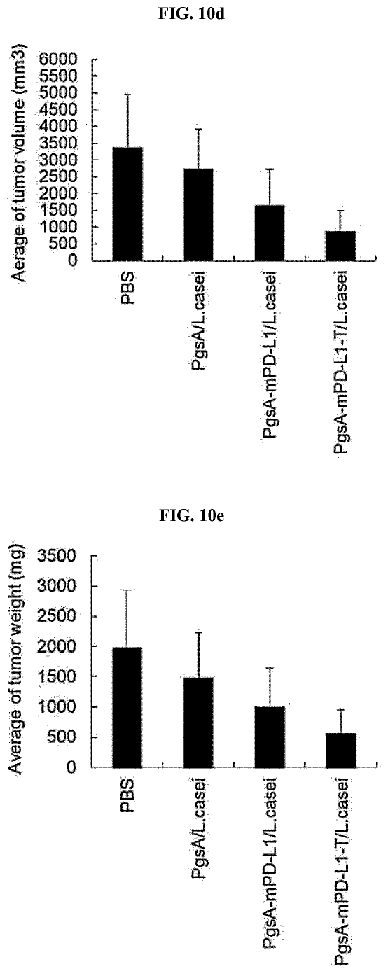

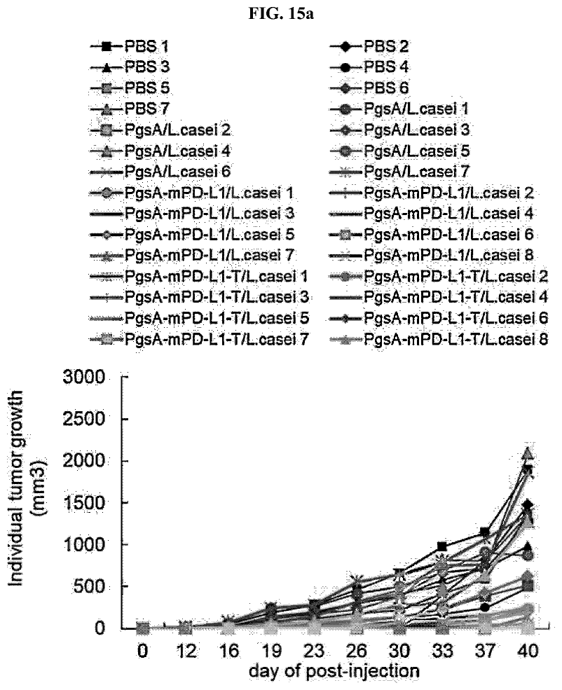

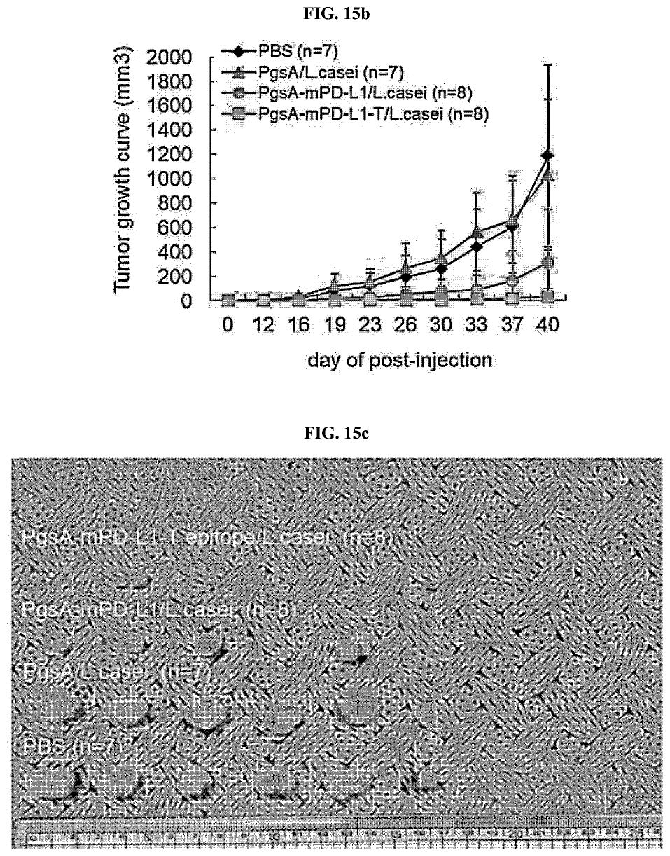

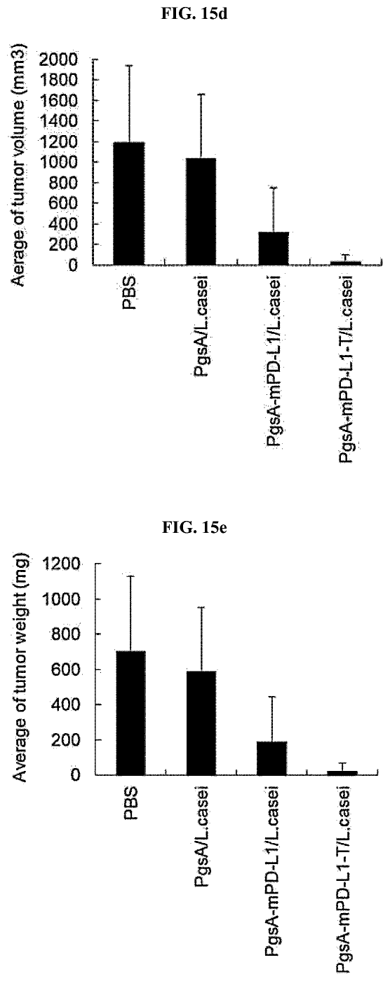

The inventors used the mPD-L1 or mPD-L1-T-epitope vaccinated mouse models of the present invention to validate the inhibitory effect on colon cancer development and growth. Specifically, on the seventh day after the third intramuscular injection of the antigens, BALB/c and C57BL/6 mice vaccinated with mPD-L1 or mPD-L1-T-epitope were subjected to subcutaneous transplantation of CT26 and MC38 mouse colon cancer cell lines into their right flanks. Colorectal cancer development and growth were monitored for 6 weeks, and when tumor development became visible and measurable, tumor sizes were measured twice a week, every 3 to 4 days, to track individual tumor growth. Also, the mean and standard deviation of tumor growth values measured per group in each mouse model were analyzed. The results showed that the growth of tumors in the PgsA-mPD-L1/L. casei group and the PgsA-mPD-L1-T epitope/L. casei group of the present invention was significantly reduced compared to the PBS and PgsA/L. casei groups. Particularly, the PgsA-mPD-L1-T epitope/L. casei group showed a more reduced growth rate compared to the PgsA-mPD-L1/L. casei group (FIGS. 10a, 10b, 15a and 15b). Additionally, the tumors were harvested from the mice 6 weeks after transplanting the colon cancer cell lines, to compare the size of the tumors. As a result, the tumors in the PgsA-mPD-L1/L. casei group and the PgsA-mPD-L1-T epitope/L. casei group of the present invention were found to be much smaller in size compared to the PBS and PgsA/L. casei groups (FIGS. 10c and 15c). In particular, the size of tumors developed and grown in the PgsA-mPD-L1-T epitope/L. casei group of the present invention was significantly smaller than the size of tumors in PgsA-mPD-L1/L. casei (FIGS. 10d, 10e, 15d and 15e).

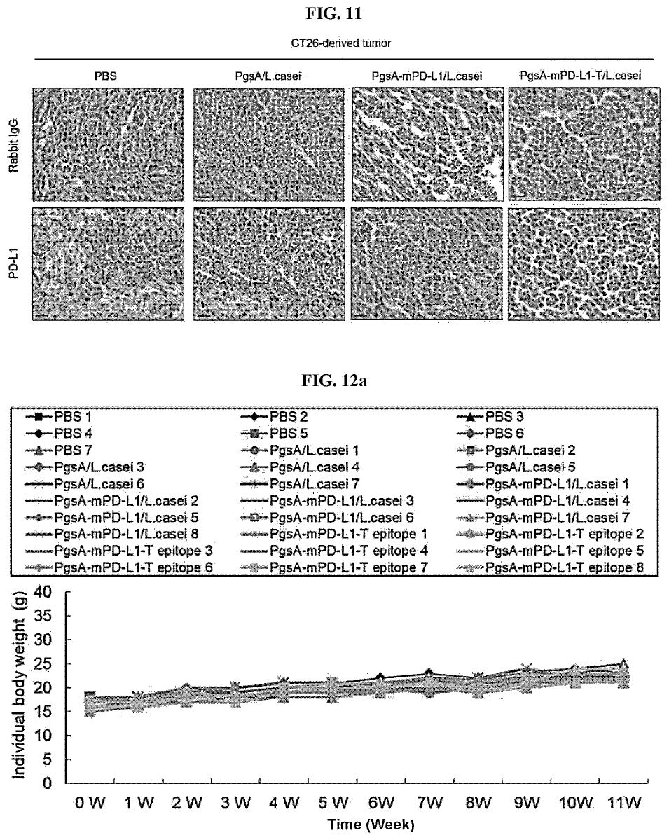

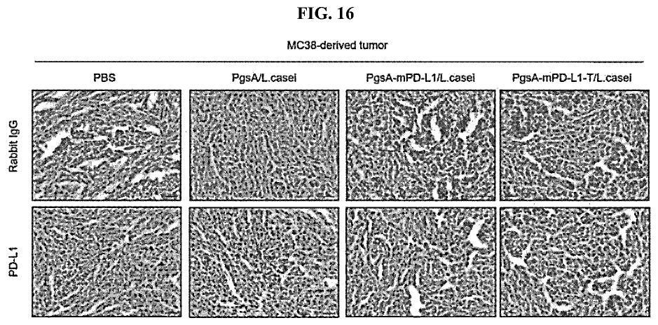

Furthermore, immunohistochemical staining of mouse PD-L1 expressed in tumor tissues harvested from the above mouse models confirmed that PD-L1 expression was significantly reduced in the PgsA-mPD-L1/L. casei group and the PgsA-mPD-L1-T epitope/L. casei group according to one embodiment of the present invention compared to the PBS and PgsA/L. casei groups (FIGS. 11 and 16). In accordance with the above results, it was found that mPD-L1 and mPD-L1-T epitope vaccination using the mouse models of the present invention inhibited the development and growth of colon cancer, and in particular, the mPD-L1-T epitope vaccination method was shown to exhibit a higher anti-cancer effect compared to the mPD-L1 vaccination method, thereby more effectively inhibiting tumor development and growth.

The above results suggest that mPD-L1 or mPD-L1-T-epitope vaccination in mouse models of the present invention is highly effective in inhibiting mouse colon cancer development and growth.

The invention has been described with reference to the embodiments described above, but these are exemplary only, and one having ordinary skill in the art will understand that various modifications and other equally valid embodiments are possible from them. Therefore, the true scope of technical protection of the invention should be determined by the technical ideas of the appended claims of the patent.

1. A recombinant bacterium transformed to present on its surface a PD-L1 protein, or a fusion protein in which the PD-L1 protein is linked to a T epitope.

2. The recombinant bacterium according to claim 1, wherein the PD-L1 protein or the fusion protein is presented on the surface by binding to a PgsA anchor.

3. The recombinant bacterium according to claim 2, wherein the PD-L1 protein or the fusion protein is inserted into a multiple cloning site of a pKV-Pald-PgsA380L vector.

4. The bacterium according to claim 1, wherein the bacterium is a non-pathogenic bacterium genus selected from the group consisting of Lactobacillus sp., Parabacteroides sp., Streptococcus sp., Bifidobacterium sp., Lactococcus sp. and Enterococcus sp.

5. The recombinant bacterium according to claim 1, wherein the PD-L1 protein comprises an amino acid sequence selected from the group consisting of SEQ ID NOs: 2 to 6 and 9.

6. An anti-cancer vaccine composition, comprising the recombinant bacterium of claim 1 as an active ingredient.

7. The anti-cancer vaccine composition according to claim 6, having anti-cancer activity against Breast cancer, Lung cancer, Colorectal cancer, Gastric cancer, Bladder cancer, Pancreatic cancer, Prostate cancer, or diffuse large B-cell lymphoma.

8. A method of preventing and treating cancer, comprising administering the anticancer vaccine composition of claim 6 to an individual suffering from cancer.

9. A method of preventing and treating cancer, comprising administering the anticancer vaccine composition of claim 7 to an individual suffering from cancer.
